(19) United States
(12) Patent Application Publication
(10) Pub. No.: US 2020/0205675 A1
(43) Pub. Date: Jul. 2, 2020

(54) SYSTEM AND METHOD FOR ENHANCING ACCURACY OF BODY SURFACE TEMPERATURE MEASUREMENT

(71) Applicant: NATIONAL CHENG KUNG UNIVERSITY, TAINAN CITY (TW)

(72) Inventors: Nai-Ying KO, KAOHSIUNG CITY (TW); Chun-Yin YEH, TAINAN CITY (TW); Wen-Chien KO, TAINAN CITY (TW); Po-Lin CHEN, TAINAN CITY (TW); Yu-Chen SHU, TAINAN CITY (TW); Kun-Ta CHUANG, TAINAN CITY (TW); Hung-Yu KAO, TAINAN CITY (TW)

(21) Appl. No.: 16/596,744

(22) Filed: Oct. 8, 2019

Related U.S. Application Data
(60) Provisional application No. 62/785,698, filed on Dec. 28, 2018.

(30) Foreign Application Priority Data
Jul. 22, 2019 (TW) 108125879

(51) Int. Cl. A61B 5/0205; A61B 5/01; A61B 5/00

(52) U.S. Cl. CPC A61B 5/02055; A61B 5/024; A61B 5/7278; A61B 5/01

(57) ABSTRACT

A system for enhancing accuracy of body surface temperature measurement includes: a core body temperature sensor configured to obtain a core body temperature data of a user, a heart rate sensor configured to obtain a heart rate data of the user, a physical indicator sensor configured to obtain at least one physical indicator of the user, a body surface temperature sensor configured to obtain a body surface temperature data of the user, an environment temperature sensor configured to obtain an environment temperature data, a humidity sensor configured to obtain an environment humidity data, and a processor. The processor is configured to calculate a representative feature temperature data of the user according to the core temperature data, the heart rate data, the at least one physical indicator, the body surface temperature data, the environment temperature data, and the environment humidity data.

### RELATED APPLICATIONS

This application claims priority to U.S. Provisional Application Ser. No. 62/785,698 filed Dec. 28, 2018, and Taiwan Application Serial Number 108125879, filed Jul. 22, 2019, the disclosures of which are incorporated herein by reference in their entireties.

### BACKGROUND

### Field of Invention

The present invention relates to a system for enhancing accuracy of body surface temperature measurement. More particularly, the present invention relates to a system and a method for enhancing accuracy of body surface temperature measurement.

### Description of Related Art

The change of body temperature of a patient is one of the significant characteristics reflecting that the patient is infected by one of plural infectious diseases, and therefore accuracy of body surface temperature measurement is very important. In general, the body surface temperature of a human body is affected by plural factor, and these factors may decrease the accuracy of body surface temperature measurement. These factors may be divided into endogenous factors and exogenous factors. The endogenous factors are, for example, age, gender, BMI (body mass index), body clock, physical activity, measurement position, etc. The exogenous factors are, for example, the environment temperature, the environment humidity, etc.

Traditionally, when measuring the body surface temperature, the aforementioned physical indicators and the aforementioned environmental factors that affect the accuracy of body surface temperature measurement are not taken into consideration, thereby resulting in poor accuracy of body surface temperature measurement. Therefore, there is a need for developing a system and a method for enhancing accuracy of body surface temperature measurement.

### SUMMARY

The present invention provides a system for enhancing accuracy of body surface temperature measurement. The system includes: a core body temperature sensor configured to obtain a core body temperature data of a user, a heart rate sensor configured to obtain a heart rate data of the user, a physical indicator sensor configured to obtain at least one physical indicator of the user, a body surface temperature sensor configured to obtain a body surface temperature data of the user, an environment temperature sensor configured to obtain an environment temperature data, a humidity sensor configured to obtain an environment humidity data, and a processor. The processor is configured to calculate a representative feature temperature data of the user according to the core temperature data, the heart rate data, the at least one physical indicator, the body surface temperature data, the environment temperature data, and the environment humidity data.

In accordance with one or more embodiments of the invention, the physical indicators include calorie consumption, a movement step number, and information of speed and displacement provided by a gravity sensor (G sensor).

In accordance with one or more embodiments of the invention, the processor calculates the representative feature temperature data based on a statistical model. The statistical model and plural parameters of the statistical model are associated with a user profile of the user.

In accordance with one or more embodiments of the invention, the user profile of the user includes at least one of age of the user, gender of the user, and an infected group of the user.

In accordance with one or more embodiments of the invention, the processor calculates the representative feature temperature data based on a linear regression model.

The present invention provides a method for enhancing accuracy of body surface temperature measurement. The method includes: obtaining a core body temperature data, a heart rate data, at least one physical indicator, a body surface temperature data of a user; obtaining an environment temperature data of an environment where the user is located; obtaining an environment humidity data of the environment where the user is located; and calculating a representative feature temperature data of the user according to the core temperature data, the heart rate data, the at least one physical indicator, the body surface temperature data, the environment temperature data, and the environment humidity data.

In accordance with one or more embodiments of the invention, the physical indicators include calorie consumption, a movement step number, and information of speed and displacement provided by a gravity sensor (G sensor).

In accordance with one or more embodiments of the invention, the representative feature temperature data is calculated based on a statistical model. The statistical model and plural parameters of the statistical model are associated with a user profile of the user.

In accordance with one or more embodiments of the invention, the user profile of the user includes at least one of age of the user, gender of the user, and an infected group of the user.

In accordance with one or more embodiments of the invention, the representative feature temperature data is calculated based on a linear regression model.

### BRIEF DESCRIPTION OF THE DRAWINGS

The invention can be more fully understood by reading the following detailed description of the embodiment, with reference made to the accompanying drawings as follows:

FIG. 1 illustrates a block diagram of a system for enhancing accuracy of body surface temperature measurement according to some embodiments of the present invention.

FIG. 2 illustrates a flow chart of a method for enhancing accuracy of body surface temperature measurement according to some embodiments of the present invention.

### DETAILED DESCRIPTION

The detailed explanation of the invention is described as following. The described preferred embodiments are presented for purposes of illustrations and description, and they are not intended to limit the scope of the invention.

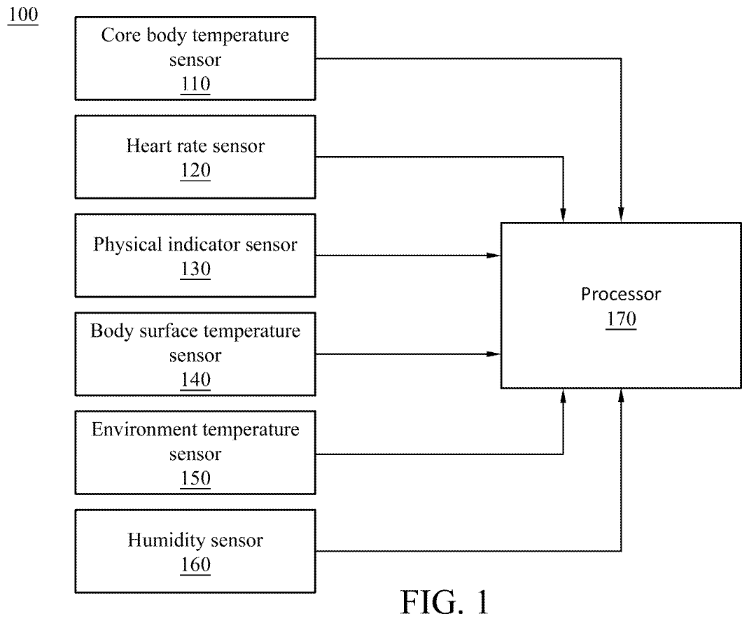

FIG. 1 illustrates a block diagram of a system 100 for enhancing accuracy of body surface temperature measurement according to some embodiments of the present invention. The system 100 includes a core body temperature sensor 110, a heart rate sensor 120, a physical indicator sensor 130, a body surface temperature sensor 140, an environment temperature sensor 150, a humidity sensor 160, and a processor 170. As shown in FIG. 1, the processor 170 is electrically connected to the core body temperature sensor 110, the heart rate sensor 120, the physical indicator sensor 130, the body surface temperature sensor 140, the environment temperature sensor 150, and the humidity sensor 160.

In some embodiments of the present invention, the core body temperature sensor 110, the heart rate sensor 120, the physical indicator sensor 130, and the body surface temperature sensor 140 may be disposed in a wearable device (e.g., a smart bracelet) worn by a user. The core body temperature sensor 110 is configured to obtain a core body temperature data of the user. The heart rate sensor 120 is configured to obtain a heart rate data of the user. The physical indicator sensor 130 is configured to obtain at least one physical indicator of the user. In some embodiments of the present invention, for example, the physical indicators may include, but not limited to, calorie consumption, a movement step number, and/or information of speed and displacement provided by a gravity sensor (G sensor), etc. The body surface temperature sensor 140 is configured to obtain a body surface temperature data of the user.

In one embodiment of the present invention, the core body temperature data obtained by the core body temperature sensor 110 includes plural core body temperatures obtained by continuous measurement. In another embodiment of the present invention, the core body temperature data obtained by the core body temperature sensor 110 includes the core body temperature measured at the current time. In one embodiment of the present invention, the heart rate data obtained by the heart rate sensor 120 includes plural heart rates obtained by continuous measurement. In another embodiment of the present invention, the heart rate data obtained by the heart rate sensor 120 includes the heart rate measured at the current time. In one embodiment of the present invention, the physical indicators obtained by the physical indicator sensor 130 are obtained by continuous measurement. For example, the physical indicators may be plural calorie consumptions obtained by continuous measurement, plural movement step numbers obtained by continuous measurement, and/or plural information of speed and displacement provided by a gravity sensor (G sensor) obtained by continuous measurement. In another embodiment of the present invention, physical indicators obtained by the physical indicator sensor 130 are measured at the current time. For example, the physical indicators may be the calorie consumption accumulated during a given time interval, the movement step number accumulated during the given time interval, and/or the information of speed and displacement provided by a gravity sensor (G sensor) accumulated during the given time interval. In one embodiment of the present invention, the body surface temperature data obtained by the body surface temperature sensor 140 includes plural body surface temperatures obtained by continuous measurement. In another embodiment of the present invention, the body surface temperature data obtained by the body surface temperature sensor 140 includes the body surface temperature measured at the current time.

In some embodiments of the present invention, the environment temperature sensor 150 may be disposed in the wearable device worn by the user or may be disposed in another sensing device electrically connected to the processor 170. The environment temperature sensor 150 is configured to obtain an environment temperature data of an environment where the user is located. In one embodiment of the present invention, the environment temperature data obtained by the environment temperature sensor 150 includes plural environment temperatures obtained by continuous measurement. In another embodiment of the present invention, the environment temperature data obtained by the environment temperature sensor 150 includes the environment temperature measured at the current time.

In some embodiments of the present invention, the humidity sensor 160 may be disposed in the wearable device worn by the user or may be disposed in another sensing device electrically connected to the processor 170. The humidity sensor 160 is configured to obtain an environment humidity data of the environment where the user is located. In one embodiment of the present invention, the environment humidity data obtained by the humidity sensor 160 includes plural environment humidities obtained by continuous measurement. In another embodiment of the present invention, the environment humidity data obtained by the humidity sensor 160 includes the environment humidity measured at the current time.

In some embodiments of the present invention, the processor 170 is, for example, a microcontroller (MCU), a central processing unit (CPU) or an application specific integrated circuit (ASIC), and is configured to calculate a representative feature temperature data of the user according to the core temperature data obtained by the core body temperature sensor 110, the heart rate data obtained by the heart rate sensor 120, the at least one physical indicator obtained by the physical indicator sensor 130, the body surface temperature data obtained by the body surface temperature sensor 140, the environment temperature data obtained by the environment temperature sensor 150, and the environment humidity data obtained by the humidity sensor 160. Specially, the body surface temperature of the human body will be affected by plural factor, while the aforementioned factors that affect the accuracy of body surface temperature measurement are already taken into consideration in the present invention, and therefore the present invention may enhance the accuracy of body surface temperature measurement.

In some embodiments of the present invention, the processor 170 calculates the representative feature temperature data based on a statistical model, and the calculation formula is shown as formula (1) as follows:

Z=F(T_{core},HR,PI,T_{surface},Tᵣₒₒₘ,Hᵣₒₒₘ) (1)

In formula (1), Z represents the representative feature temperature data of the user; T_{core} represents the core temperature data; HR represents the heart rate data; PI represents the physical indicators; T_{surface} represents the body surface temperature data; Tᵣₒₒₘ represents the environment temperature data; Hᵣₒₒₘ represents the environment humidity data obtained by the humidity sensor 160; and F represents the statistical model. In some embodiments of the present invention, the statistical model adopted by the processor 170 may be a linear regression model or another suitable statistical model. In some embodiments of the present invention, the statistical model is implemented by software and/or hardware, and integrated in the processor 170 (e.g., stored in a nonvolatile memory of the processor 170). Specifically, the statistical model adopted by the processor 170 and plural parameters of the statistical model are associated with a user profile of the user. In some embodiments of the present invention, the aforementioned user profile of the user includes at least one of age of the user, gender of the user, and an infected group of the user. It is worth mentioning that the statistical model adopted by the processor 170 and the parameters of the statistical model may vary depending on the infected group of the user (different infectious diseases). It is worth mentioning that the statistical model adopted by the processor 170 and the parameters of the statistical model may also vary depending on whether a user of a healthy group is in active status or in rest status (not in active status). For example, in one embodiment of the present invention, for the user of the healthy group in rest status (not in active status), the representative feature temperature data of that user may be obtained by the following calculation formula (2):

Z=T_{surface}+0.64×sex−0.03×age+0.71×Tᵣₒₒₘ+0.31×Hᵣₒₒₘ−0.17×T_{core}−0.01×HR (2)

In formula (2), age represents the age of the user; sex represents the gender of the user. When the gender of the user is male, sex=1, and when the gender of the user is female, sex=0.

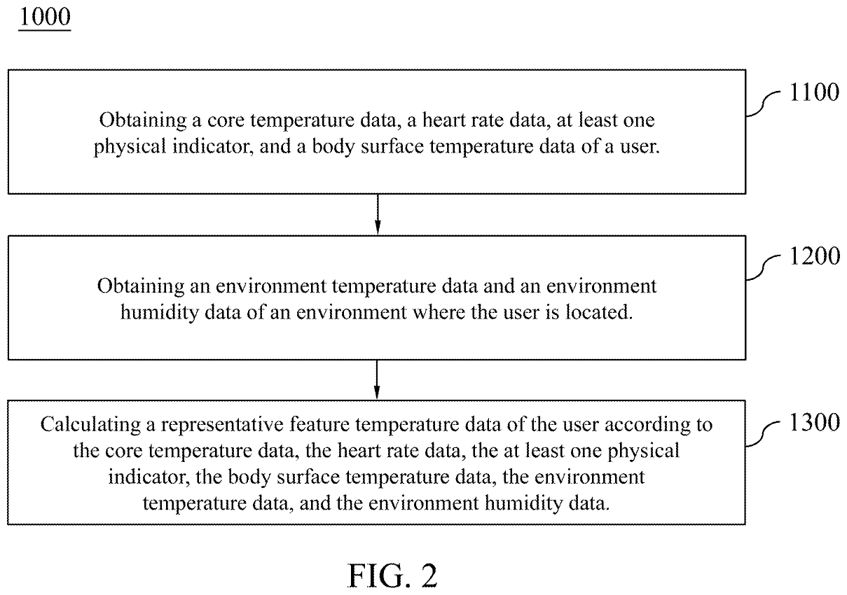

FIG. 2 illustrates a flow chart of a method 1000 for enhancing accuracy of body surface temperature measurement according to some embodiments of the present invention. Referring to FIGS. 1 and 2, the method 1000 includes steps 1100, 1200, and 1300. In step 1100, the core temperature data of the user is obtained by the core body temperature sensor 110; the heart rate data of the user is obtained by the heart rate sensor 120, the at least one physical indicator of the user is obtained by the physical indicator sensor 130, the body surface temperature data of the user is obtained by the body surface temperature sensor 140. In step 1200, the environment temperature data of an environment where the user is located is obtained by the environment temperature sensor 150, and the environment humidity data of the environment where the user is located is obtained by the humidity sensor 160. In step 1300, the process 170 calculates the representative feature temperature data of the user according to the core temperature data, the heart rate data, the at least one physical indicator, the body surface temperature data, the environment temperature data, and the environment humidity data. Details regarding the steps 1100, 1200, and 1300 have been discussed in detail in the above description of the system 100 for enhancing accuracy of body surface temperature measurement, and are not repeated here.

From the above description, the present invention provides the system 100 and the method 1000 for enhancing accuracy of body surface temperature measurement. The physical indicators and the environmental factors that affect the accuracy of body surface temperature measurement are taken into consideration when calculating the body surface temperature, thereby enhancing accuracy of body surface temperature measurement.

Although the present invention has been described in considerable detail with reference to certain embodiments thereof, other embodiments are possible. Therefore, the spirit and scope of the appended claims should not be limited to the description of the embodiments contained herein. It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A system for enhancing accuracy of body surface temperature measurement, comprising:
a core body temperature sensor configured to obtain a core body temperature data of a user;
a heart rate sensor configured to obtain a heart rate data of the user;
a physical indicator sensor configured to obtain at least one physical indicator of the user;
a body surface temperature sensor configured to obtain a body surface temperature data of the user;
an environment temperature sensor configured to obtain an environment temperature data;
a humidity sensor configured to obtain an environment humidity data; and
a processor configured to calculate a representative feature temperature data of the user according to the core temperature data, the heart rate data, the at least one physical indicator, the body surface temperature data, the environment temperature data, and the environment humidity data.

2. The system of claim 1, wherein the physical indicators comprise calorie consumption, a movement step number, and information of speed and displacement provided by a gravity sensor (G sensor).

3. The system of claim 1, wherein the processor calculates the representative feature temperature data based on a statistical model, wherein the statistical model and a plurality of parameters of the statistical model are associated with a user profile of the user.

4. The system of claim 3, wherein the user profile of the user comprises at least one of age of the user, gender of the user, and an infected group of the user.

5. The system of claim 1, wherein the processor calculates the representative feature temperature data based on a linear regression model.

6. A method for enhancing accuracy of body surface temperature measurement, comprising:
obtaining a core body temperature data, a heart rate data, at least one physical indicator, and a body surface temperature data of a user;
obtaining an environment temperature data of an environment where the user is located;
obtaining an environment humidity data of the environment where the user is located; and
calculating a representative feature temperature data of the user according to the core temperature data, the heart rate data, the at least one physical indicator, the body surface temperature data, the environment temperature data, and the environment humidity data.

7. The method of claim 6, wherein the physical indicators comprise calorie consumption, a movement step number, and information of speed and displacement provided by a gravity sensor.

8. The method of claim 6, wherein the representative feature temperature data is calculated based on a statistical model, wherein the statistical model and a plurality of parameters of the statistical model are associated with a user profile of the user.

9. The method of claim 8, wherein the user profile of the user comprises at least one of age of the user, gender of the user, and an infected group of the user.

10. The method of claim 6, wherein the representative feature temperature data is calculated based on a linear regression model.
